(19) United States
(12) Patent Application Publication
(10) Pub. No.: US 2009/0216128 A1
(43) Pub. Date: Aug. 27, 2009

(54) Broadband Ultrasonic Probe

(75) Inventor: Armen P. Sarvazyan, Lambertville, NJ (US)

Correspondence Address: BORIS LESCHINSKY, P.O. BOX 72, WALDWICK, NJ, 07463, US

(73) Assignee: ARTANN LABORATORIES, INC., Lambertville, NJ (US)

(21) Appl. No.: 12/036,531

(22) Filed: Feb. 25, 2008

(51) Int. Cl. A61B 8/14

(52) U.S. Cl. 600/459

(57) ABSTRACT

An ultrasound probe includes a reverberator having a randomly uneven shape preferably including a plurality of facets. Together with a rigid coupling window, it forms a closed cavity filled with reverberation medium such as water. One or more ultrasound transducers are placed inside the reverberator to generate a signal using time-reversed acoustics principles. Additional transducers increase the power output of the probe. An optional transducer design features a piezomaterial formed in a randomly uneven shape, preferably having length/thickness ratio of at least 2. The reverberator cavity further includes scatterers suspended inside and aimed at improving the focusing quality of the probe. Such scatterers can be of various sizes and in a number of shapes such as beads, cylinders and membranes. The probe can be advantageously used for focusing broadband ultrasonic waves in various industrial and medical applications such as those utilizing high intensity short ultrasonic pulses.

### FIELD OF THE INVENTION

The present invention relates generally to an apparatus for generating and focusing broadband ultrasonic waves for various industrial and medical applications. More specifically, it relates to medical imaging, surgery and therapy devices employing high intensity short ultrasonic pulses.

### BACKGROUND OF THE INVENTION

The bandwidth of the probes generating ultrasonic waves for various medical and industrial applications is mainly defined by the bandwidth of the transducers employed in the probe. These transducers are typically made in the form of discs and plates of piezoelectric materials of certain constant thickness, which defines main resonance frequency of the transducer. The piezoelectric materials typically used are lead zirconate titanate (PZT) piezoceramic, polyvinylidene difluoride (PVDF) piezopolymer films, and PZT ceramic/polymer composite. The most widely used commercial piezoelectric material is PZT. These PZT and other piezoceramic transducers are of narrow bandwidth because they have relatively high acoustic impedance and without a complex damping and matching techniques they cannot induce broadband signals. This limits applicability of piezoceramic transducers in the cases when a broadband acoustical signal should be generated and, more specifically, when a short pulses need to be generated. Generation and reception of short ultrasonic pulses are important medical imaging applications and in certain applications in industrial Non Destructive Evaluation (NDE) of materials. Short high power ultrasonic pulses are required in therapeutic applications of ultrasound, such as lithotripsy—ultrasonic disintegration of kidney and bladder stones.

Increasing the bandwidth of a piezoceramic transducer by damping significantly affects their efficiency. Therefore piezoceramic ultrasound transducers used in applications, which require short pulses with broad spectral content, such as in medical imaging, suffer from the trade-off between bandwidth and sensitivity, which impedes the optimization of ultrasound image quality. Another disadvantage of piezoceramic transducers is high acoustic impedance which is significantly larger than that of the human body and needs additional matching layers for medical applications.

To increase the bandwidth of the transducers, as well as to provide better matching with biological tissue, there have been proposed so-called piezoelectric composites in which piezoelectric ceramic material is surrounded by a piezoelectrically passive polymer matrix. The combination of a piezoelectric ceramic with a polymer, such as silicon rubber or epoxy, results in a material having low acoustic impedance, good piezoelectric properties and provides significantly broader frequency band being employed in transmitting and receiving transducers. Ultrasonic transducers made of a piezoelectric composite of such structure that a number of ceramic piezoelectric poles are buried in a plate-like polymer matrix perpendicular to the plate surface is disclosed in the U.S. Pat. No. 4,683,396 to Takeuchi et al.

The piezocomposite structures most commonly used in ultrasonic transducers are the 1-3 and 2-2 structures. Transducers with 1-3 structure comprise a multiplicity of mutually-parallel, spaced apart PZT rods embedded in a matrix of conformal polymer filler material, while the 2-2 structure comprises alternating layers of piezoelectric ceramic and polymer. Due to the effect of the polymer filler material damping, the piezocomposites provide a broader bandwidth.

A broadband ultrasonic transducers technology, which has some similarity to piezocomposites, called Multidomain Ultrasonic Transducer (MUT) has been recently reported (Ostrovskii I. “Acousto-domain interaction in ferroelectric lithium niobate.” J Acoust Soc. Am, 2004, 115: 2456.) The MUT is a two-dimensional multidomain ferroelectric vibrator, in which an array of inversely poled ferroelectric domains is incorporated. Typical materials for the MUT are LiNbO₃ and LiTaO₃, or other materials with high dielectric constant and piezoelectric modulus. MUT irradiates an acoustic wave along its axis into an adjacent media, with which the surface is brought into close acoustical contact.

Another type of a broadband transducer that can be used as an effective receiver as well as transmitter especially for high frequencies tasks is based on piezoelectric polymers such as PVDF (Ohigashi, H. “Ultrasonic Transducers in the Megahertz Range,” in The Applications of Ferroelectric Polymers, Ed., T. T. Wang, J. M. Herbert and A. M. Glass, Chapman and Hall, New York, 1988 and Chen Q. X. and P. A. Payne, “Industrial Applications of Piezoelectric Polymer Transducers,” Measurement Sciences and Technology, Vol. 6, 1995, pp. 249-267). The low acoustic impedance of this polymer makes it attractive to medical applications of ultrasonic imaging. The transducer can be coupled directly to the patients' skin and provide an effective sound transmission to the test area. Other forms of making PVDF transducers include the use of aluminum backing. Aluminum has relatively low acoustic impedance as compared to other widely used metals and a direct backing of aluminum enables to form an effective broadband ultrasonic transducer.

Although both composite and piezopolymer transducers have wider bandwidth than conventional piezoceramics, it is still insufficient for some applications, which need radiation of very narrow ultrasonic pulse signals. In addition, PVDF transducers are ineffective at the frequencies below 1 MHz while that frequency range is important for certain therapeutic applications based on the cavitational mechanisms of bioeffects.

The need exists therefore for a broadband transducer capable of radiating short ultrasonic pulses as well accurately focusing acoustic energy in the region of interest inside the body.

Focusing of short ultrasonic pulses is a fundamental aspect of most medical applications of ultrasound. In ultrasonic imaging, the quality of acoustic waves focusing is directly related to such important parameter of imaging as spatial resolution. In therapeutic and surgical applications, effective focusing of ultrasound is important for delivering sufficient amount of acoustic energy to a target tissue to achieve necessary biological effect as well as for selective action on the lesion, which needs to be treated, all without damaging surrounding healthy tissues.

Conventional approaches of ultrasound focusing include geometrical focusing and electronic focusing. Geometrical focusing is based on the use of concave piezoceramic elements manufactured as a part of a spherical shell or acoustic lenses made commonly from a solid material with the sound velocity, which is higher than that of a water-like media (O'Neil, H. T. Theory of focusing radiators. J. Acoust. Soc. Am. 1949, 21, 516-526). Geometrical focusing systems are simple, inexpensive and easy to make, but their principal disadvantage is that they have a fixed focal distance and could not steer the focus along and off the axis.

Electronic focusing is based on the use of phased-array systems consisting of a number of separate elements (Ebbini E. S., Cain C. A. A spherical-section ultrasound phased-array applicator for deep localized hyperthermia. IEEE Trans. Biomed. Eng. 1991 V. 38. No 7. P. 634-643). These elements are energized from their own power amplifiers and allow changing in controllable way the phase relationships over the array aperture therefore creating any desired shape of a wave front. Such arrays permit to steer a focus along and off the axis of the array.

An alternative technique of focusing ultrasonic waves is based on principles of Time-Reversed Acoustics (TRA) as first described by Fink, M., 1997, “Time Reversed Acoustics,” Physics Today, March 1997, pp. 34-40, which is incorporated herein by reference in its entirety. The TRA technique is based on the reciprocity of acoustic propagation, which implies that the time-reversed version of an incident pressure field naturally refocuses on its source. U.S. Pat. No. 5,092,336 to Fink, which is also incorporated herein by reference in its entirety, describes a device for localization and focusing of acoustic waves in tissues.

Several examples of TRA ultrasound focusing systems are described in the U.S. patent application Ser. No. 10/370,134 (US Patent Application Publication No. 2004/0162550) and U.S. patent application Ser. No. 10/370,381 (US Patent Application Publication No. 2004/0162507) to Govari et al. as well as a European Patent Application No. EP1449564, all of which are incorporated herein by reference in their entirety.

The present invention relates to a broadband probe for focusing ultrasound waves utilizing the TRA principle. In medicine, the present invention may be used most advantageously with TRA methods and devices designed for various diagnostic and therapeutic ultrasound and other acoustic wave applications including cavitational destruction of tissues, ultrasound imaging and image-guided interventions, ultrasonic lithotripsy, ultrasound-assisted drug delivery, and ultrasonic surgery. High intensity focused ultrasound (HIFU) recently became an effective and widespread medical therapy technique. An expected benefit of HIFU is the creation of a clinical effect in a desired, confined location within a body, without damage to intervening tissue. Therefore, proper focusing and control is one of the primary criteria for successful therapeutic application of ultrasound.

There is a long-felt need for a probe, which combines the advantages of wide bandwidth, efficient focusing ability, good matching with biological tissue, and the ability to provide high intensity ultrasonic pulse.

### SUMMARY OF THE INVENTION

The ultrasonic probe of the present invention may be used advantageously as part of medical ultrasonic instruments both for imaging and therapy as described below in more detail.

Accordingly, it is an object of the present invention to overcome the above-mentioned and other drawbacks of the prior art by providing a novel device for generating and focusing broadband ultrasonic signals using time-reversal principle.

The invention broadly describes an ultrasound probe having a reverberator and an ultrasound transducer suspended therein to generate an ultrasound wave signal. The probe of the invention includes a reverberator placed or formed within a rigid housing and having a randomly uneven shape, preferably incorporating a plurality of facets. Together with a coupling window, the reverberator forms a closed cavity filled with reverberation medium such as preferably water. One or more ultrasound transducers are placed inside the reverberator or in alternative embodiments incorporated on or within a wall of the reverberator and are adapted to generate a TRA signal when driven appropriately by an electronic unit. Additional transducers increase the power output of the probe. Novel design features of the transducers include using a piezomaterial formed in a randomly uneven shape, preferably with the length to thickness ratio of at least 2. When such transducers are immersed in the reverberation medium, provisions are proposed to insulate the transducers from that medium by having protective coatings thereon.

Additional advantageous features of the probe of the invention include the presence of scatterers suspended inside the reverberator and aimed at improving the focusing quality of the probe. Such scatterers can be of various sizes and in a number of shapes, for example beads, cylinders and membranes.

### BRIEF DESCRIPTION OF THE DRAWINGS

Other characteristics and advantages of the invention will become apparent in the course of the following description of several of its embodiments, given by way of non-limiting examples, in conjunction with the appended drawings.

FIG. 1A presents a schematic diagram of the time-reversal focusing system with an embodiment of the probe of the invention with a broadband inner transducer.

FIG. 1B presents another embodiment of the probe of the invention, where the reverberation chamber 5 is made as an irregular cavity inside a solid piece of metal 17.

FIG. 2 illustrates the details of the broadband transducer.

FIG. 3 presents a schematic of a probe configuration using several inner broadband transducers.

FIG. 4 presents a schematic of a probe configuration with a broadband transducer mounted in the wall of the reverberator.

FIG. 5 presents an embodiment of the probe of the invention comprising and a set of internal scatterers.

FIG. 6 presents an embodiment of the probe of the invention with a faceted reverberator having internally attached transducers with one flat side.

FIG. 7 presents an embodiment of the probe of the invention encompassing a faceted reverberator with externally attached transducers having one flat side and a set of internal scatterers.

FIG. 8 presents an embodiment of the probe of the invention with a faceted reverberator made from a solid material such as aluminum and with externally attached transducers having one flat side.

### DETAILED DESCRIPTION OF THE PREFERRED EMBODIMENTS OF THE INVENTION

A detailed description of the present invention follows with reference to accompanying drawings in which like elements are indicated by like reference letters and numerals.

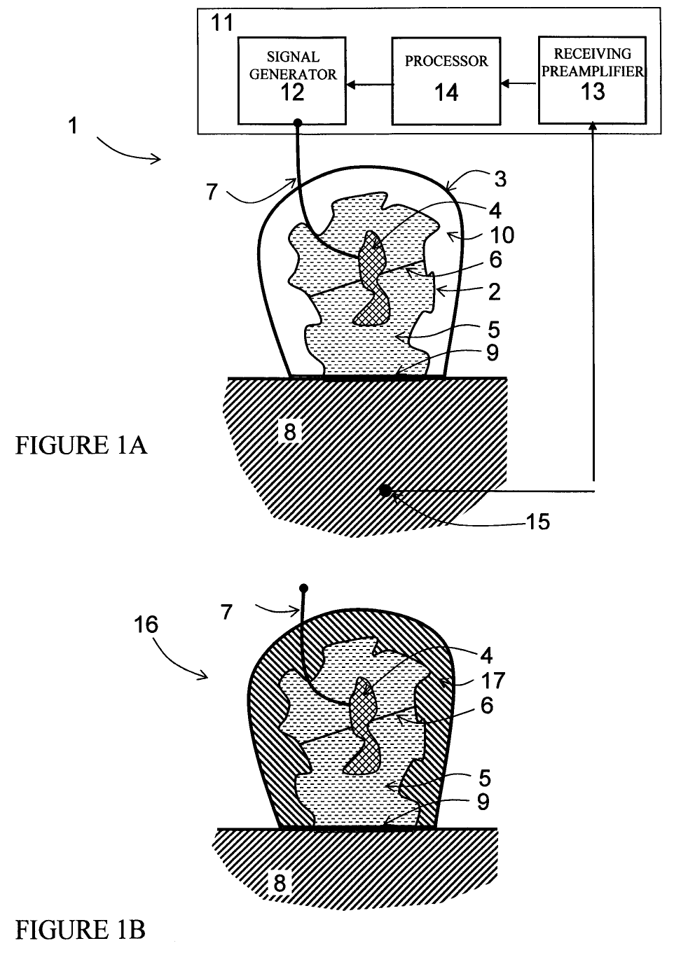

FIG. 1A illustrates a general concept of the TRA system 1 with a broadband focusing probe. The probe includes a reverberator 2 forming a closed cavity together with a coupling window 9. Reverberator 2 is enclosed in an outer housing 3, while a transducer 4 is placed into the reverberation medium 5 filling the reverberator 2. The transducer 4 is firmly attached to the walls of the reverberator 2 by a plurality of supporting pins 6 or other fixation means while being suspended therewithin and being in contact with the reverberation medium. It is further connected by a cable means 7 to a signal generator 12 of the time-reversal electronic unit 11. In addition to the signal generator 12, the electronic unit 11 comprises a receiving preamplifier 13 and a processor 14.

To be able to focus ultrasound onto a particular target area 15, the TRA system needs to be tuned by performing the following procedure. The signal generator 12 first generates the initial excitation signal and applies it to the TRA transducer 4. A beacon/hydrophone 15 is placed in the target focal area 15 and receives the acoustical signal, which is then transmitted to be amplified by a preamplifier 13, time-reversed and stored in the memory of the processor 14. After that procedure the TRA system is ready to generate an ultrasonic beam focused on the target area 15 by applying the stored time-reversed electrical signal to the transducer 4.

The reverberation medium 5 could be preferably water or an aqueous solution or any other fluid with low absorption of the acoustic waves. Most of organic and inorganic liquids have higher ultrasound absorption coefficient than water or aqueous solutions and will be less efficient in accumulating acoustic energy in time, which is an important feature of the TRA technology. Another advantage of water or an aqueous solution is that it ensures a good acoustic coupling with the biological tissue 8 because tissue is composed of 60%-80% of water so the acoustical parameters of tissue are close to that of water.

The reverberator 2 is made with preferably thin walls surrounded by cavity 10. The space 10 between these walls of the reverberator 2 and the outer housing 3 is filled with air, which provides conditions for better reflection of ultrasound from the reverberator walls to prevent acoustic energy to escape from the reverberation medium 5. Good reflection of acoustic waves is necessary for efficient time reversal focusing. In fact, reducing the air pressure in the cavity 10 and applying vacuum thereto may further improve the performance of the probe.

As shown in FIG. 1A, the exterior wall surface of the reverberator 2 is made to have a randomly uneven shape. It preferably but not necessarily includes a plurality of facets of different sizes with dimensions preferably in the range corresponding to 1-10 wavelengths of ultrasound. The word “facet” for the purposes of this description includes a portion of the internal surface of the reverberator, which may be flat or curved. The randomness and multiplicity of facets of the reverberator enhances the focusing ability of the TRA device of the invention. The inner surface of the walls of reverberator 2 is further made rough with irregularities in the range of a fraction of ultrasound wavelength, preferably in the range of 0.01 to 1 wavelength of ultrasound so that it acts not only as a reflector but also as a scatterer for acoustic wave. This feature further enhances the TRA focusing ability of the probe. The boundary of the reverberation medium includes a coupling window 9 providing an acoustical contact with biological tissue. It is made of a sufficiently hard material to prevent deformation when the device is placed in contact with the tissue, such as rigid polymer, glass or metal. To ensure reliable acoustical contact between the probe and the tissue a coupling gel may be used.

FIG. 1B shows another embodiment of the probe 16 in which the reverberator 5 is formed as an irregular cavity inside a solid piece of metal 17. The reflection from the boundaries of such a reverberator 2 may be acceptable for the goals of the TRA focusing because of significant mismatch in the acoustical impedances of the metal and the liquid filling the reverberator. In comparison with the probe shown in FIG. 1A, an advantage of the embodiment shown in FIG. 1B is that it is more robust and easier to manufacture.

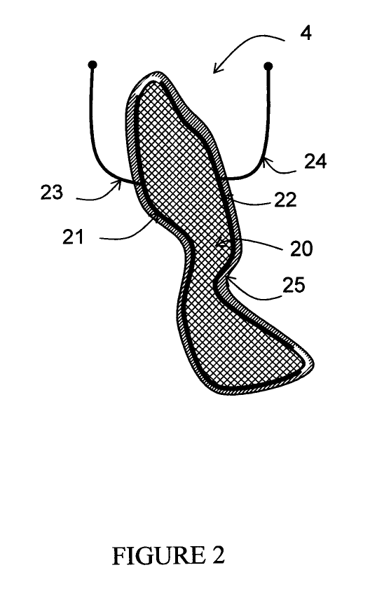

FIG. 2 shows the transducer 4 of FIG. 1 in more detail. In contrast to conventional plane-parallel transducers having well defined resonance frequency depending on their thickness, the transducer 4 has a randomly uneven shape and therefore has much broader bandwidth. The variation of the transducer length to thickness ratio should preferably be more than 2-3 fold. The body 20 of transducer 4 can be made of any conventional piezomaterial: piezoelectric crystal, piezoceramic, piezocomposite or a piezopolymer like PVDF that provides more efficient acoustic coupling with water. The surface of the transducer body 20 is covered by electrodes 21 and 22 connected by wires 23 and 24 to the output of the time reversal electronic unit (not shown on this figure). If the reverberation media is a conducting liquid, such as water, the electrodes need to be covered by an isolation coating 25, such as a layer of epoxy or silicone to prevent direct contact between metal and water and avoid corrosion.

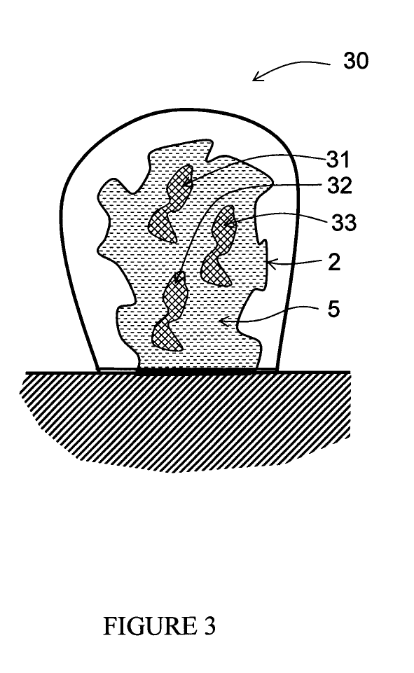

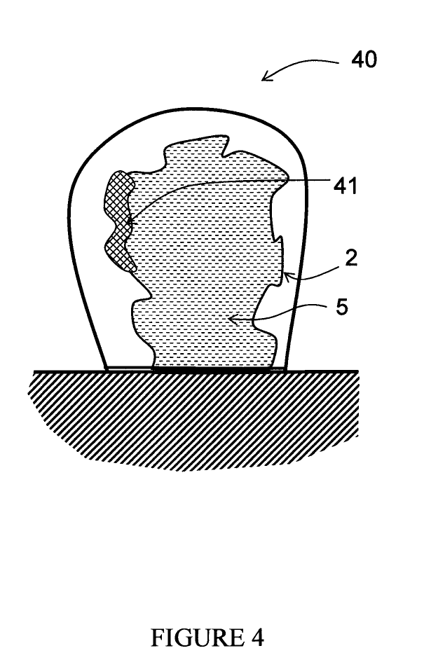

In case when high intensity of ultrasound is needed, more than one transducer can be inserted in the reverberation medium as it is illustrated in FIG. 3. An embodiment of the probe 30 illustrated in FIG. 3 is similar to that shown in FIG. 1A, except the number of the transducers is more than one. An example of a probe with 3 transducers 31, 32, 33, is shown. The transducers are firmly suspended and supported inside the inner chamber of the reverberator 2 in a manner similar to that shown on FIG. 1A. The number and the dimensions of transducers are limited by the available volume of the reverberator. Each transducer is preferably connected to a corresponding dedicated channel of the output amplifier of the TRA electronic unit (not shown in the figure). Alternatively, all transducers can be connected in parallel and powered by just one channel of the output amplifier.

FIG. 4 shows yet another embodiment of the probe 40 where a transducer 41 is incorporated in the wall of the reverberator rather than being suspended within the volume thereof. Preferably, the side of the transducer, which is exposed to the reverberation medium 5 is the ground electrode, therefore no isolation coating is needed, such as that illustrated in FIG. 2. Alternatively, the transducer can be attached to the wall of the reverberator from the outside and will have no direct contact with the reverberation medium 5. Just one or many transducers can be incorporated in the walls of the reverberator. The upper limit for number of transducers is defined by the available surface of the reverberator wall.

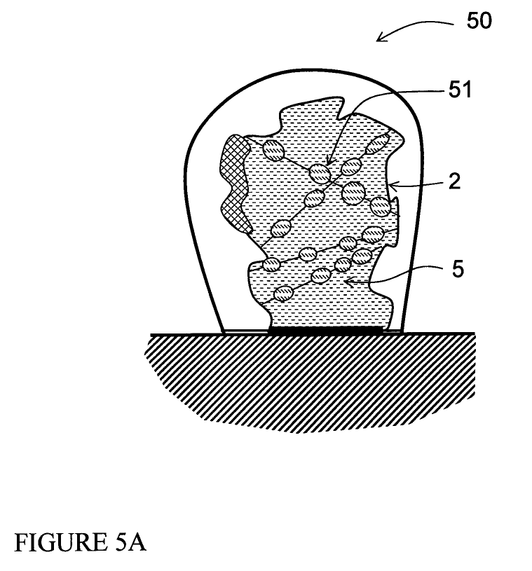

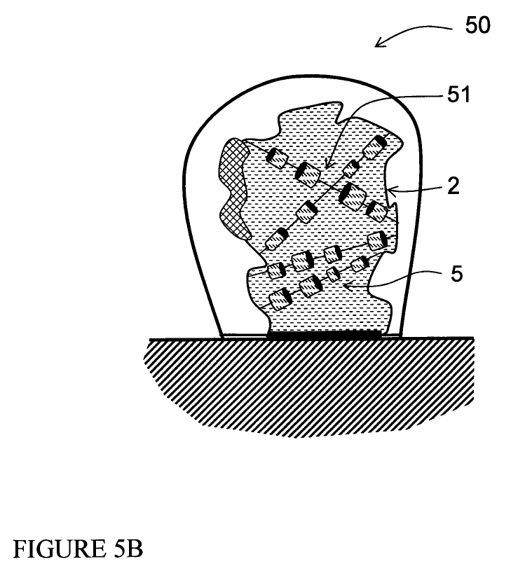

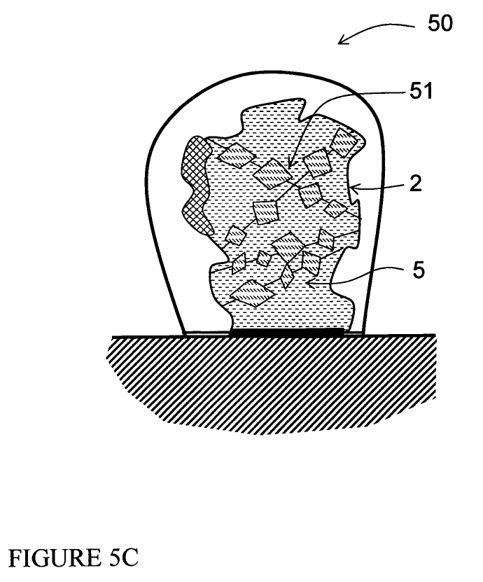

To enhance reverberation ability of reverberator 2, additional reflectors and scatterers can be incorporated in the reverberation medium, as illustrated in FIG. 5. The probe 50 shown in FIG. 5 includes several sets of the bead-shaped scatterers 51 firmly connected to the walls of the reverberator 2. The scatterers are made of a material providing high acoustic mismatch with the reverberating medium 5, such as metal, glass and alike and preferably made with highly polished surface. The scatterers can be made in various shapes and dimensions, such as beads of different configuration and size, cylinders and membranes.

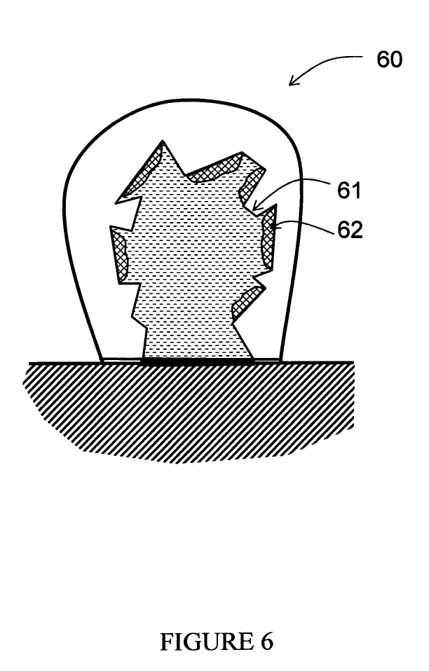

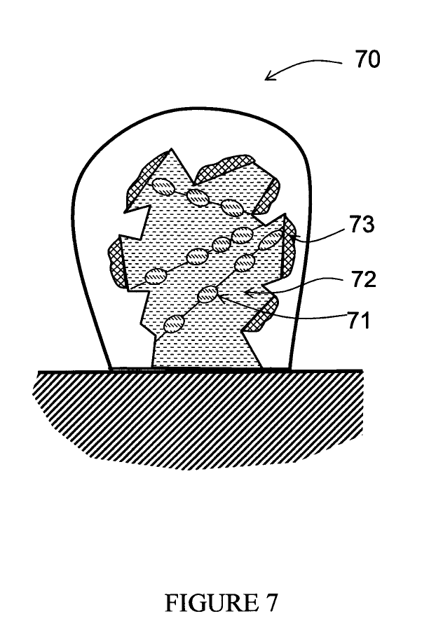

A further alternative embodiment of the probe 60 is shown in FIG. 6. The reverberator 61 is formed with facets being flat, which are randomly oriented and are sized to accept transducers 62. Transducers 62 for this embodiment should have one flat surface corresponding in size to the corresponding facet of the reverberator 2, which is advantageous from the manufacturability point of view. The number of transducers can vary from 1 up to the total number of facets of the reverberator. If higher intensity of focused ultrasound is needed, a probe can be built comprising additional transducers inserted in the reverberation medium similar to that implemented in the embodiment 30 depicted in FIG. 3. In case of the reverberator with faceted walls, the transducers can be attached to the reverberator 2 also from outside, which could be advantageous from the point of view of easier wiring. Each transducer is connected to a corresponding channel of the output amplifier of the TRA electronic unit (not shown in the figure). Alternatively, all transducers can be connected in parallel and powered by just one channel output amplifier. To further improve focusing ability of the additional scatterers and reflectors can be incorporated in the reverberation liquid of the probe similar to that shown in FIG. 6. FIG. 7 shows a schematic cross-sectional diagram of such a probe 70 having both a set of scatterers 71 firmly fixed inside the reverberation liquid 72 and a set of external transducers 73.

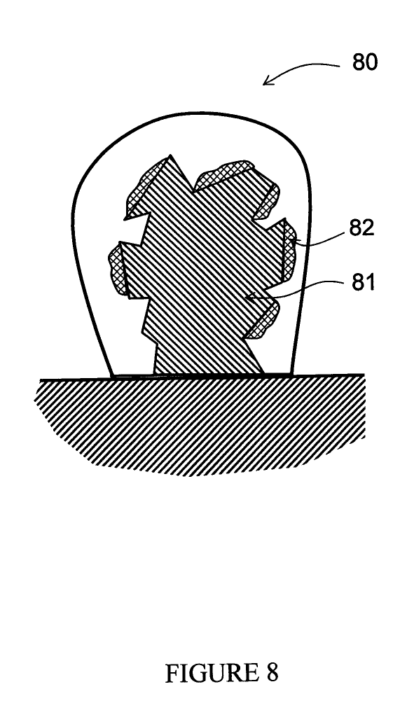

The reverberator with faceted walls can be manufactured from a solid material with low attenuation of ultrasound, such as aluminum. FIG. 8 shows a probe 80 with the reverberator 81 machined from aluminum. A set of transducers 82 is affixed to the facets of the reverberator 81.

The transducers used in the probes of FIGS. 6-8 can be made from different piezomaterials, such as piezoceramic, piezopolymer or ceramic/polymer composite. Although variable thickness transducers are preferable for generating broadband short signals, standard plane-parallel transducers can also be used with the multifaceted reverberators shown in FIGS. 6-8 in the cases where longer signals are focused and the bandwidth is not critical for a particular application.

Although the invention herein has been described with respect to particular embodiments, it is understood that these embodiments are merely illustrative of the principles and applications of the present invention. It is therefore understood that numerous modifications may be made to the illustrative embodiments and that other arrangements may be devised without departing from the spirit and scope of the present invention as defined by the appended claims.

1. A broadband ultrasonic probe for a time-reversal acoustic focusing system, said probe comprising:
a reverberator enclosed in an outer housing including a rigid coupling window, said reverberator together with said coupling window forming a closed fixed geometry cavity filled with a reverberation medium, said reverberator having a randomly uneven asymmetrical shape, and
at least one ultrasonic transducer exposed to said reverberation medium, said at least one transducer equipped with a cable connecting thereof to a signal generator of the time-reversal acoustic system.

2. The probe as in claim 1, wherein said shape of reverberator includes a plurality of facets.

3. The probe as in claim 1, wherein said reverberator is separated from said outer housing by a space filled with air.

4. The probe as in claim 2, wherein said facets of said reverberator are selected to have dimensions in the range of about one to ten times a wavelength of ultrasound signal as generated by the time-reversal acoustic focusing system.

5. The probe as in claim 1, wherein said reverberator has an inner surface made with irregularities having a size in a range from about 0.01 to about 1 times a wavelength of ultrasound signal as generated by the time-reversal acoustic focusing system.

6. The probe as in claim 1, wherein said reverberation medium is water.

7. The probe as in claim 1, wherein said transducer is suspended within said reverberator and affixed thereto.

8. The probe as in claim 1, wherein said transducer is attached to a wall of said reverberator.

9. (canceled)

10. The probe as in claim 1, wherein said probe comprises a plurality of ultrasound transducers exposed to said reverberation medium.

11. The probe as in claim 1, wherein said probe further comprises at least one scatterer of ultrasound exposed to said reverberation medium.

12. The probe as in claim 1, wherein said probe further comprises a plurality of scatterers of ultrasound contained within said reverberator.

13. The probe as in claim 12, wherein said plurality of scatterers includes scatterers of different shapes and sizes.

14. The probe as in claim 13, wherein said shapes of said scatterers are selected from a group consisting of beads, cylinders, and membranes defined as thin sheets.

15. The probe as in claim 1, wherein said transducer has a randomly uneven shape.

16. The probe as in claim 15, wherein said transducer has a ratio of length to thickness of at least two fold.

17. The probe as in claim 1, wherein said at least one transducer has an isolation coating on an outer surface thereof to insulate it from the surrounding reverberation medium.

18. The probe as in claim 2, wherein said facets of said reverberator are flat, said transducer is shaped to have a flat surface dimensioned to mate with a corresponding facet of said reverberator, said transducer is affixed to said reverberator along said facet.
